SEQUENCE LISTING (5 sequences)

SEQ ID NO: 1 - DNA, 27 bp, Artificial
  Common forward primer
ggagggacggaagaaagagtgaaggga

SEQ ID NO: 2 - DNA, 27 bp, Artificial
  WT Reverse Primer
cgagctcatagattatataagcacctg

SEQ ID NO: 3 - DNA, 27 bp, Artificial
  WT Probe
gaggataaacaaacgggaccatagaag

SEQ ID NO: 4 - DNA, 24 bp, Artificial
  DAS-591227-7 Reverse Primer
ccttaattctccgctcatgatcag

SEQ ID NO: 5 - DNA, 26 bp, Artificial
  DAS-591227-7 Probe
tttaaactgaaggcgggaaacgacaa

(19) United States
(12) Patent Application Publication
(10) Pub. No.: US 2014/0017684 A1
(43) Pub. Date: Jan. 16, 2014

(54) METHODS TO DETERMINE ZYGOSITY IN A BULKED SAMPLE

(71) Applicant: Chandra-Shekara A. Channabasavaradhya, Carmel, IN (US)

(72) Inventor: Chandra-Shekara A. Channabasavaradhya, Carmel, IN (US)

(73) Assignee: DOW AGROSCIENCES LLC, Indianapolis, IN (US)

(21) Appl. No.: 13/977,432

(22) PCT Filed: Dec. 28, 2011
(86) PCT No.: PCT/US11/67503

§ 371 Date: Sep. 26, 2013

Related U.S. Application Data
(60) Provisional application No. 61/428,142, filed on Dec. 29, 2010.

(51) Int. Cl. C12Q 1/68

(52) U.S. Cl. CPC C12Q 1/6895
USPC 435/6.11

(57) ABSTRACT

Methods of determining the presence or absence of an inserted nucleotide sequence at a particular insertion site in a nucleic acid include: isolating a nucleic acid from the bulked tissue sample; contacting the nucleic acid with a forward primer able to bind to the nucleic acid upstream of the insertion site, a first reverse primer specific for the inserted nucleotide sequence, and a second reverse primer able to bind to the nucleic acid downstream of the insertion site. The primers may be used to reproduce nucleic acids between the primers. The reproduced nucleic acids may be analyzed to determine if an inserted nucleotide sequence is present or absent in the sample.

### CROSS-REFERENCE TO RELATED APPLICATION

This application is a national phase entry under 35 U.S.C. §371 of International Patent Application PCT/US2011/067503, filed Dec. 28, 2011, designating the United States of America and published in English as International Patent Publication WO 2012/092327 A2 on Jul. 5, 2012, which claims the benefit under Article 8 of the Patent Cooperation Treaty and under 35 U.S.C. §119(e) to U.S. Provisional Patent Application Ser. No. 61/428,142, filed Dec. 29, 2010, the disclosure of each of which is hereby incorporated herein by this reference in its entirety.

### BACKGROUND OF THE INVENTION

Quality control testing for any contamination in a finished line is very critical for successful hybrid seed production and maintaining and building good business relationship with the customers. The contamination during seed increase of a finished line may come from pollination of unintended transgenic or non-transgenic plants grown near the production site or during seed processing and most importantly, contamination due to pollen leakage from sterile plants. To ensure that the finished line is completely homozygous and free from any hemizygous, null or unintended transgenic lines, tester-row method is currently being followed. The tester-row method utilizes ELISA technology to estimate zygosity status based on protein levels on an individual plant basis. Since the assay is based on single plant basis, it is very time consuming as well as expensive. In addition, there is an additional cost of carrying out tester-row method in the field. The ELISA method is useful in detecting silencing of the transgene expression by detecting the protein level. It is not sensitive and robust to detect any hemizygous, null or any other unintended contamination in the finished seed lot. In addition, when ELISA method is used, a separate tissue sampling is required for adventitious presence testing.

### BRIEF SUMMARY OF THE INVENTION

Particular embodiments of the invention include methods of determining the presence or absence of an inserted nucleotide sequence at a particular insertion site in a nucleic acid. Embodiments may comprise: isolating nucleic acid from the bulked sample; contacting the nucleic acid with a forward primer able to bind to the nucleic acid upstream of the insertion site, and a reverse primer able to bind to the nucleic acid downstream of the insertion site. The primers may be used to reproduce nucleic acids between the primers. The reproduced nucleic acids may be analyzed to determine if an inserted nucleotide sequence is present or absent in a bulked sample.

Embodiments may comprise: isolating nucleic acid from the sample; contacting the nucleic acid with a forward primer able to bind to the nucleic acid upstream of the insertion site, and a reverse prime able to bind to the nucleic acid downstream of the insertion site. The primers may be used to reproduce nucleic acids between the primers in the first portion and second portion. The reproduced nucleic acids may be analyzed to determine if inserted nucleotide sequence is present or absent in the sample. A second reaction either multiplexed with the above reaction or as a singleplex can be carried out using a forward primer and a reverse primer that detects an endogenous gene or sequence. This second reaction can be used as an internal control to determine the quality and quantity of the DNA and/or PCR conditions used.

### BRIEF DESCRIPTION OF THE DRAWINGS

FIG. 1A is a schematic representation of the elements for a method of determining the presence or absence of an inserted nucleotide sequence at a particular insertion site according to an embodiment of the invention. Therein, the possibly inserted nucleotide sequence (110) is represented by the dark block, while the surrounding genome (120) is indicated by the open segments. Also depicted are forward primer (130), first reverse primer (140), and second reverse primer (150). Further represented are optional insert-specific probe (160) and wild-type-specific probe (170).

FIG. 1B illustrates a modified assay that includes making a standard zygosity protocol into two separate reactions: Reaction 1 including a common primer, and wild-type-specific primer, and a wild-type-specific probe (FAM); and Reaction 2 including endogenous control (Invertase1 gene) with VIC probe.

FIG. 2A is a schematic representation of a first replicated product (200) according to an embodiment of the invention. Also depicted are forward primer (130), first reverse primer (140), and optional insert-specific probe (160).

FIG. 2B is a schematic representation of a first replicated product (200) according to an embodiment of the invention. Also depicted are forward primer (130), second reverse primer (150), and optional wild-type-specific probe (170).

FIG. 3 is a schematic representation of the elements for a method of determining the presence or absence of an inserted nucleotide sequence at a particular insertion site according to an embodiment of the invention. A first reaction (400) involves the possibly inserted nucleotide sequence (110) that is represented by the dark block, while the surrounding genome (120) is indicated by the open segments. Also depicted are forward primer (130), first reverse primer (140), and optional insert-specific probe (160).

A second reaction (500) involves the possibly inserted nucleotide sequence (110) that is represented by the dark block, while the surrounding genome (120) is indicated by the open segments. Also depicted are forward primer (130), second reverse primer (150), and optional wild-type-specific probe (170).

FIG. 4 is graphical representation of FAM fluorescence results from a Roche LIGHTCYCLER® 480.

FIG. 5 is graphical representation of VIC fluorescence results from a Roche LIGHTCYCLER® 480.

### DETAILED DESCRIPTION OF THE INVENTION

Embodiments of the invention include methods of determining the presence or absence of an inserted nucleotide sequence at a particular insertion site in a sample of nucleic acids. In some embodiments, the nucleic acids may be isolated and/or purified from a single source or a population of sources, which population may include one or more individuals which may or may not each of have distinct nucleic acids. In other embodiments, the source of nucleic acids may be, but is not limited to, animal, plant, bacteria, archaea, protists, fungi, protozoa, chromistae, eukaryotic, prokaryotic, in vivo, in vitro, cell, seed, gamete, maize, soy, wheat, rape, rice, and generated sources.

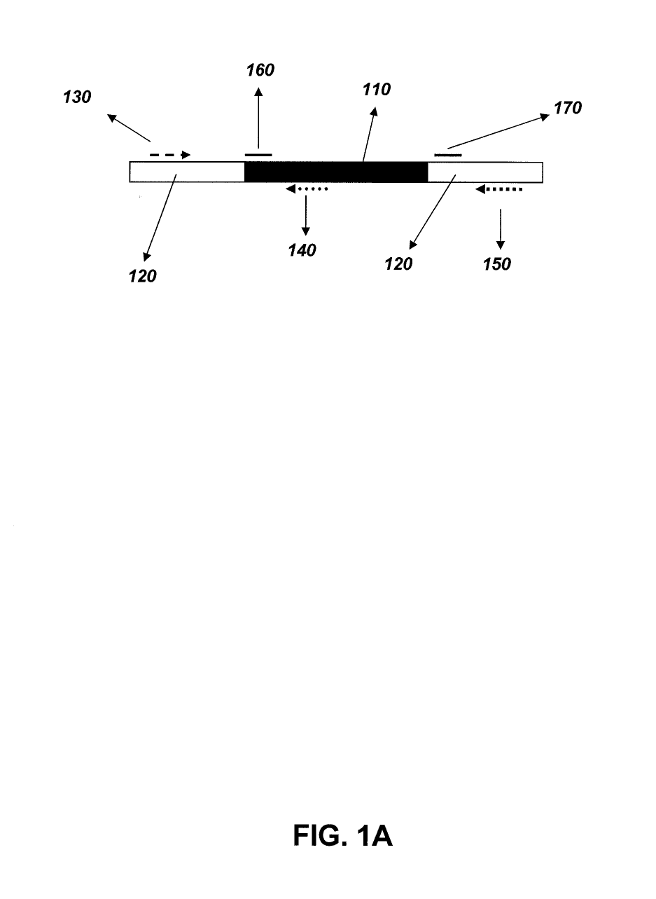

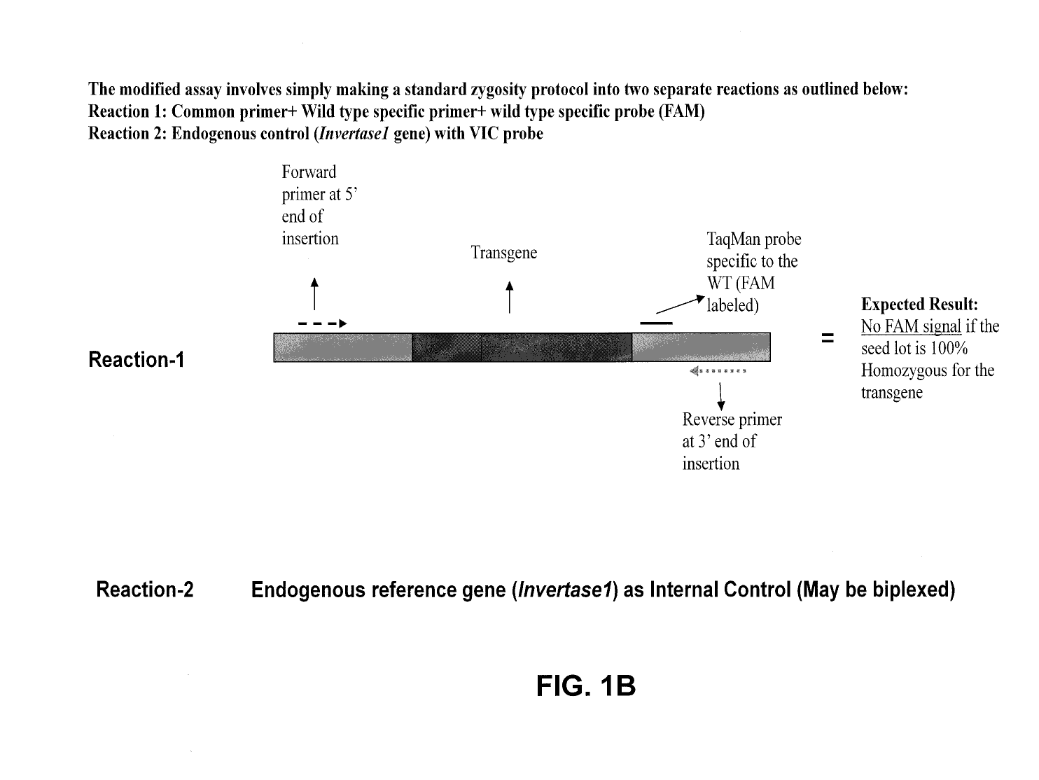

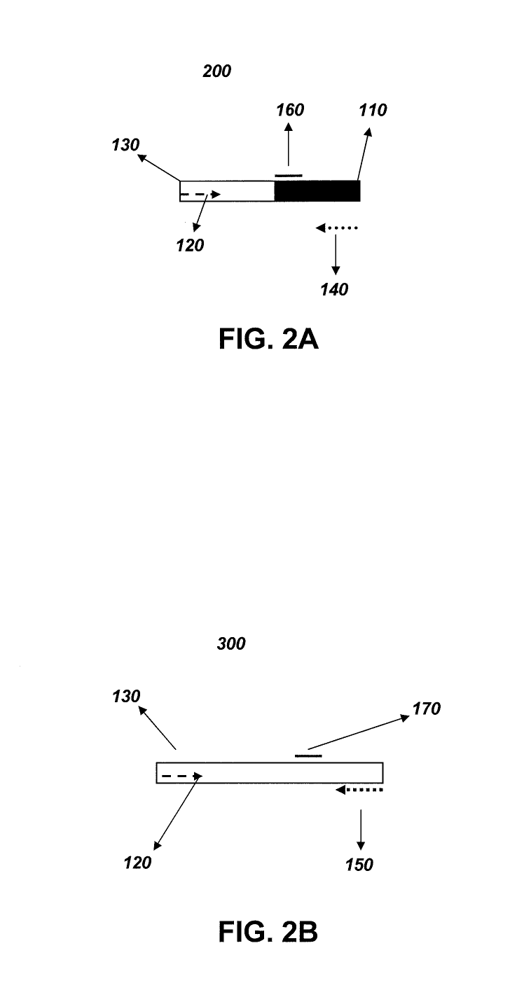

In particular embodiments, the method may comprise obtaining, isolating, purifying, and/or partially purifying nucleic acid. With reference to FIG. 1, the isolated nucleic acids may be contacted with a forward primer (130) able to bind to the nucleic acid upstream of the insertion site (120) and a first reverse primer (140) capable of specifically binding to sequence within the inserted nucleotide sequence (110) (if present), and a second reverse primer (150) capable of specifically binding to a sequence downstream (120) of the insertion site and allowing the primers to anneal to the isolated nucleic acids. The intervening sequences between the primers may then be reproduced, if possible, using the primers to primer replication, via techniques well known in the art, such as, but not limited to, Polymerase Chain Reaction (PCR). For insertion sites where the inserted nucleotide sequence (110) is present and larger than a fragment reproducible by standard methods (e.g. >5 kb), products of the reproduction can include a first replicated product (FIG. 2A (200)) comprising those sequences between the forward primer (130) and the first reverse primer (140) but may generally lack a second replicated product (FIG. 2B (300)) primed from the second reverse primer (150). For insertion sites where the inserted nucleotide sequence is not present, the products of the reproduction can include the second replicated product (FIG. 2B (300)) comprising those sequences between the forward primer (130) and the second reverse primer (150). Where the inserted nucleotide sequence (110) is present at some, but not all of the insertion sites in the nucleic acid, a mixture of the two products will result. The results of the reproduction are then analyzed to determine the presence and/or relative levels of the first replicated product (200) and/or the second replicated product (300).

For insertion sites where the inserted nucleotide sequence is present, the products of the reproduction of the nucleic acid may include the first replicated product (FIG. 2A (200)) comprising those sequences between the forward primer (130) and the first reverse primer (150). For insertion sites where the inserted nucleotide sequence is not present, the products of the reproduction may include the second replicated product (FIG. 2B (300)) comprising those sequences between the forward primer (130) and the second reverse primer (150). Where the inserted nucleotide sequence (110) is present at some, but not all of the insertion sites in the nucleic acid, a mixture of the two products will result. The results of the reproduction are then analyzed to determine the presence and/or relative levels of the first replicated product (200) and/or the second replicated product (300).

In other embodiments, in the presence of the inserted nucleotide sequence at the insertion site, the forward primer and the first reverse primer will be less than approximately 5 kb apart and the forward primer and the second reverse primer will be more than approximately 5 kb apart. In further embodiments, wherein the inserted nucleotide sequence is absent from the insertion site, the forward primer and the second reverse primer will be less than approximately 5 kb apart.

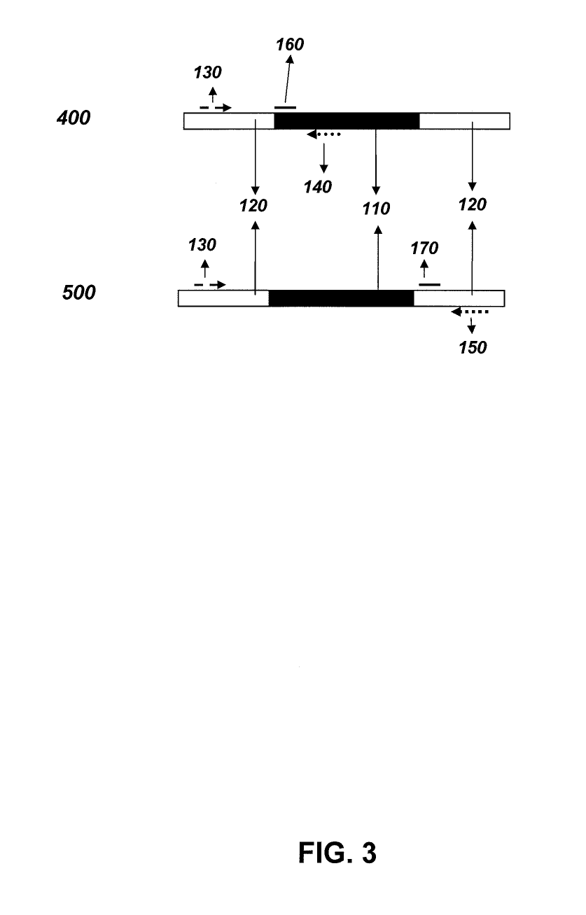

In particular embodiments, with reference to FIG. 3, the isolated nucleic acid may be divided into several portions. A first portion of the nucleic acid may be contacted with a forward primer (130) able to bind to the nucleic acid upstream of the insertion site (120) and a first reverse primer (140) capable of specifically binding to sequence within the inserted nucleotide sequence (110) (if present), and allowing the primers to anneal to the isolated nucleic acids. The intervening sequences between the primers may then be reproduced, if possible, using the primers to primer replication, via techniques well known in the art, such as, but not limited to PCR. For insertion sites where the inserted nucleotide sequence (110) is present, products of the reproduction can include a first replicated product (FIG. 2A (200)) comprising those sequences between the forward primer (130) and the first reverse primer (140).

A second portion of the nucleic acid may be contacted with a forward primer (130) able to bind to the nucleic acid upstream of the insertion site (120) and a second reverse primer (150) capable of specifically binding to a sequence downstream (120) of the insertion site, and allowing the primers to anneal to the isolated nucleic acid. The intervening sequences between the primers may then be reproduced, if possible, using the primers to prime replication, via techniques well known in the art, such as, but not limited to PCR. For insertion sites where the inserted nucleotide sequence (110) is not present, products of the reproduction may include a second replicated product (FIG. 2B (300)) comprising those sequences between the forward primer (130) and the second reverse primer (150).

For insertion sites where the inserted nucleotide sequence is present, the products of the reproduction of the first portion of the nucleic acid may include the first replicated product (FIG. 2A (200)) comprising those sequences between the forward primer (130) and the first reverse primer (150). For insertion sites where the inserted nucleotide sequence is not present, the products of the reproduction of the second portion of the nucleic acid may include the second replicated product (FIG. 2B (300)) comprising those sequences between the forward primer (130) and the second reverse primer (150).

In other embodiments, the methods may be used to detect the presence of the insert in the nucleic acid where the insert is present in less than 10%, 9%, 8%, 7%, 6%, 5%, 4%, 3%, 2%, or 1% of the insertion sites in the nucleic acids. In embodiments, the methods may be used to detect the absence of the insert in the nucleic acid where the insert is absent in less than 10%, 9%, 8%, 7%, 6%, 5%, 4%, 3%, 2%, or 1% of the insertion sites in the nucleic acids.

In some embodiments, the nucleic acid containing the insertion site may be any kind of nucleic acid including, but not limited to, DNA, RNA, PNA, or other modified forms of nucleic acids.

In further embodiments, the presence and/or amounts of the first and/or second replication product may be detected by any means known in the art such as, but not limited to, insert-specific probes (160) and wild-type-specific probes (170) respectively. In particular embodiments, a probe may be a nucleotide sequence capable of binding at a specific site in the first and/or the second replication product. The annealing of a probe may take place during or after replication.

By way of non-limiting examples, the presence and/or amounts of the first and/or second replication product may be detected through the use of chromatography, gels, labels, moieties, southern blots, and northern blots.

In certain embodiments, fluorophore may be attached to one or more the probes to ease detection. Additionally, a fluorophore quenching molecule may also be attached to the probe. Examples of such probes containing a fluorophore and a fluorophore quenching molecule include the TAQMAN® system and reagents available from Roche Molecular Diagnostics and/or Applied Biosystems. In other embodiments, the production and levels of the first and/or second reproduction products may be monitored in real time.

In some embodiments, the methods described herein may be used to determine zygosity of nucleic acids (e.g. genome(s)) at a particular insertion site. Results of the assays wherein the first replicated product (200) is present and second replication product (300) is absent indicate that the nucleic acids are homozygous for the presence of the insert. Results of the assays wherein the first replicated product (200) is absent and second replication product (300) is present indicate that the nucleic acids are homozygous for the absence of the insert. Results of such assays wherein both first replication product (200) and second replication product (300) are present indicate that the nucleic acids are heterozygous for the insert (e.g. at least one insertion site contains the insert and at least one insertion site does not contain the insert).

In particular embodiments, sets of primers and/or probes may be combined with one or more other sets of primers and probes so as to allow the detection of the presence or absence one or more inserts within one or more particular insertion sites in the nucleic acid of a sample. As used herein, a “set of primers and/or probes” includes at least one forward primer able to bind to a site upstream of a particular insertion site and at least one reverse primer able to bind to a site downstream of a particular insertion site or within a particular insertion. In other embodiments, multiple sets of primers and/or probes may be used to detect a particular insert at one or more particular insertion sites and/or multiple inserts at multiple particular insertion sites.

In certain embodiments, methods described herein may be used to screen a population for the presence or absence of an insertion at a particular insertion site. In further embodiments, the presence of a particular insertion site may be determined for each member of the population.

As used herein, “particular insertion site” denotes a known location or conserved sequence within a nucleic acid where an insert may be reproducibly inserted. In certain embodiments, the presence of a particular insertion site may be determined for each nucleic acid in a sample, by way of non-limiting example, through the production of a first (200) or a second (300) replicated product by the methods described herein. In other embodiments, the sequences flanking the particular insertion site or sequences within the insert may be conserved. In further embodiments such conservation in the sequences flanking the particular insertion site or within the insert may be limited to the binding sites of primers and/or probes. As used in this context, “conserved” denotes that a specific primer and/or probe is able to specifically bind to the area that is “conserved.” In particular embodiments, the specific primer and/or probe will remain bound to the “conserved” area under highly stringent conditions.

As used herein, “upstream” and “downstream” are relative terms and designate opposite sides of an insertion site in a nucleic acid. Which direction is located “upstream” and “downstream” of an insertion site is not denoted by the terms, only that they lie on opposite sides of the insertion site.

As used herein, “forward primer” and “reverse primer” are relative terms denoting primers binding to differing locations on a nucleic acid so as to enable the reproduction of the nucleic acids between them by methods available in the art, such as, but not limited to, the PCR. Where a particular primer is bound to a nucleic acid sequence site is not denoted by the terms “forward” and “reverse,” only that they lie on opposite sides of the sequence to be reproduced and can act as primers for a polymerase in the reproduction.

As used herein, “comprising,” “including,” “containing,” “characterized by,” and grammatical equivalents thereof are inclusive or open-ended terms that do not exclude additional, unrecited elements or method steps, but also includes the more restrictive terms “consisting of” and “consisting essentially of.”

The present invention is further described in the following examples, which are offered by way of illustration and are not intended to limit the invention in any manner.

### EXAMPLES

### Example 1

### Plant Material

Parental lines screening: To determine if the border sequence at the transgene insertion site is highly conserved and that the event-specific primers may be used across various genetic backgrounds, a total of 92 diverse inbred lines were screened that represented different heterotic groups and locations such as North America, South America, Europe, stiff stalk, non-stiff stalk, public and proprietary sources (Table 1).

TABLE 1
List of materials used for screening border sequence at the
transgene insertion site.

| | Heterotic | |
|---|---|---|
| Material type | Group | Origin |
| Proprietary | Lancaster | North American |
| Proprietary | Flint | European |
| Proprietary | Stiff stalk | North American |
| Proprietary | Mixed | South America |
| Proprietary | Lancaster | North American |
| Proprietary | Lancaster | North American |
| Proprietary | Stiff stalk | North American |
| Proprietary | Stiff stalk | North American |
| Proprietary | Stiff stalk | North American |
| Proprietary | Lodent | North American |
| Proprietary | Lodent | North American |
| Public | Stiff stalk | North American |
| Proprietary | Stiff stalk | North American |
| Proprietary | Stiff stalk | North American |
| Proprietary | Lodent | North American |
| Proprietary | Non-stiff stalk | North American |
| Proprietary | Lancaster | North American |
| Proprietary | Stiff stalk | North American |
| Proprietary | Stiff stalk | North American |
| Public | Mixed | North American |
| Proprietary | Lodent | North American |
| Proprietary | Flint | South America |
| Proprietary | Lancaster | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Mixed | South America |
| Proprietary | Mixed | South America |
| Proprietary | Mixed | South America |
| Proprietary | Mixed | North American |
| Proprietary | Mixed | South America |
| Proprietary | Mixed | South America |
| Public | Stiff stalk | North American |
| Public | Stiff stalk | North American |
| Proprietary | Lodent | North American |
| Proprietary | Lodent | North American |
| Proprietary | Stiff stalk | North American |
| Proprietary | Stiff stalk | North American |
| Proprietary | Stiff stalk | North American |
| Proprietary | Stiff stalk | North American |
| Proprietary | Lodent | North American |
| Proprietary | Lodent | North American |
| Proprietary | Tropical | South America |
| Proprietary | Tropical | South America |
| Proprietary | Tropical | South America |
| Proprietary | Tropical | South America |
| Proprietary | Lancaster | North American |
| Proprietary | Lodent | North American |
| Proprietary | Stiff stalk | North American |
| Proprietary | Non-stiff stalk | North American |
| Proprietary | Lodent | North American |
| Proprietary | Lancaster | North American |
| Proprietary | Lancaster | North American |
| Proprietary | Lancaster | North American |
| Proprietary | Lancaster | North American |
| Proprietary | Lancaster | North American |
| Proprietary | Lancaster | North American |
| Proprietary | Lancaster | North American |
| Proprietary | Lancaster | North American |
| Proprietary | Stiff stalk | North American |
| Proprietary | Stiff stalk | North American |
| Public | Lancaster | North American |
| Proprietary | Lodent | North American |
| Proprietary | Tropical | South America |
| Proprietary | Non-stiff stalk | North American |
| Proprietary | Stiff stalk | North American |
| Proprietary | Non-stiff stalk | North American |
| Public | Non-stiff stalk | North American |
| Proprietary | Lancaster | North American |
| Proprietary | Lancaster | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Stiff stalk | North American |
| Proprietary | Mixed | North American |
| Proprietary | Suwan | South America |
| Proprietary | Tropical | South America |
| Proprietary | Tropical | South America |
| Proprietary | Non-stiff stalk | North American |
| Proprietary | Mixed | North American |
| Proprietary | Mixed | South America |
| Proprietary | Mixed | South America |
| Proprietary | Tropical | South America |
| Proprietary | Mixed | South America |
| Proprietary | Mixed | North American |
| Proprietary | Lodent | North American |
| Proprietary | Lodent | North American |

Bulked Seed Screening: For demonstrating the application of DNA-based testing on a bulked seed samples and to determine sensitivity of detection, seed pools mentioned below were created using known pure homozygous DAS-59122, hemizygous DAS-59122, and null (conventional) seeds. These were counted into six different 50 mL falcone tubes:

1. 100 Homozygous DAS-59122 seeds, replication-1

2. 100 Homozygous DAS-59122 seeds, replication-2

3. 99 Homozygous DAS-59122 seeds, and one Hemizygous DAS 59122 seed, replication-1

4. 99 Homozygous DAS-59122 seeds, and one Hemizygous DAS 59122 seed, replication-2

5. 99 Homozygous DAS 59122 seeds and one null (conventional) seed, replication-1

6. 99 Homozygous DAS 59122 seeds and one null (conventional) seed, replication-2

### Example 2

### Seed Grinding and DNA Extraction

The seeds were finely ground and genomic DNA was isolated using the Qiagen DNEASY® kit (Valencia, Calif.). Five separate genomic DNA extractions were completed from each seed lot. The purified genomic DNA was quantitated using the QuantIt Picogreen DNA kit and diluted to standardized concentrations.

### Example 3

### TAQMAN®-Based Zygosity Assays

The zygosity analysis was carried out using different sets of reagents which consisted of different primer and probe sequences. The method and the reagents were designed specifically for the DAS-59122 event. A schematic of the zygosity assay design is provided in FIG. 1. The method utilized a gene-specific primer, a wild-type primer and a gene-specific/wild-type (common) primer in addition to two probes. The probes consisted of a wild-type-specific and a transgenic-specific probe. The first method incorporated all of the primers and probes within the same reaction (“single reaction method”). To increase the sensitivity of the zygosity detection, an additional method was also tested wherein two separate independent reactions were performed (FIG. 3) (“multiple reaction method”). One set of wells contained the wild-type-specific primer, common primer, and wild-type-specific probe. The other set of wells contained the transgene-specific primer, common primer, and transgene-specific probe. For example, in this method only two primers and one probe were used in a 384-well plate format in which one quadrant contained the wild-type-specific primer+common primer+wild-type-specific probe, another quadrant contained the transgene-specific primer+common primer+transgene-specific probe.

Modified End-Point TAQMAN®: A master mix containing the following components was prepared: water, 15.35 μl; 10×PCR Buffer, 2.50 μl; 25 mM MgCl₂,1.50 μl; 10 mM dNTP (2.5 mM each), 2.0 μl; 20 μM common forward primer (SEQ ID NO:1), 0.25 μl; 20 μM wild-type reverse primer (SEQ ID NO:2), 0.25 μl; 10 μM wild-type dual-labeled probe (SEQ ID NO:3) labeled with VIC at the 3′ end and BHQ2 at the 5′ end, 0.20 μl; HotStar Taq (5 U/μl), 0.20 μl; and, 10 ng/μl Genomic DNA, 3.0 μl.

A second master mix containing the following components was prepared: water, 15.35 μl; 10×PCR Buffer, 2.50 μl; 25 mM MgCl₂, 1.50 μl; 10 mM dNTP (2.5 mM each), 2.0 μl; 20 μM common forward primer (SEQ ID NO:1), 0.25 μl; 20 μM 591227 reverse primer (SEQ ID NO:4), 0.25 μl; 10 μM 591227 dual-labeled probe (SEQ ID NO:5) labeled with FAM at the 3′ end and BHQ1 at the 5′ end, 0.20 μl; HotStar Taq (5 U/μl), 0.20 μl; and, 10 ng/μl Genomic DNA, 3.0 μl.

Both cocktails were pipetted into single wells and amplified using a GenAmp PCR System 9700 for the following conditions: 95° C. for 15 minutes (1 cycle); 95° C. for 15 seconds, 60° C. for 60 seconds (35 cycles). The fluorescent readings were analyzed and zygosity was determined from the excitation of the VIC or FAM fluorophore.

Results: After determining the conserved nature of border sequences at the site of primer binding, primers were designed and tested on bulked seed pools using a standard TAQMAN® zygosity assay. The results indicated that the single reaction method was sensitive to detect null seed contamination in a pure homozygous bulked seeds with 1% sensitivity of detection. However, the single reaction method was inconsistent in detecting any hemizygous seed contamination at a level of 1% presence. The insensitivity could be due to preferential amplification of reaction-1 (see FIG. 1) due to abundant template availability. To overcome this resource competition during a PCR reaction, the single reaction method was modified into multiple separate reactions (FIG. 3). This modification resulted in the selective amplification of only the intended region without any resource competition. Use of the multiple reaction method (parent seed zygosity testing) on the parental “seed pools” proved consistent in detection contamination of both hemizygous as well as conventional (null) seed in a pure homozygous seed bulk at 1% sensitivity of detection (Table 2). The results were also confirmed through real-time PCR using Roche's LIGHTCYCLER® 480 to ensure there were no artifacts in the PCR reaction or set up.

TABLE 2
Sensitivity of detection by various zygosity methods.

| | | Consistency of | |
|---|---|---|---|
| | Consistency of | detection at 1% | Consistency of detection |
| | detection at 0% | hemizygous seed | at 1% null seed |
| Method | contamination | contamination | contamination |
| single | Yes | Yes | Inconsistent |
| reaction | | | |
| method | | | |
| multiple | Yes | Yes | Yes |
| reaction | | | |
| method | | | |

Parental Lines Screening: The event-specific primers can be used across various genetic backgrounds, 92 diverse inbred lines (Table 1) that represent different heterotic groups grown in locations such as North America, South America, and Europe were used to test the multiple reaction method. These lines were tested and confirmed to be free of transgene contamination using the protocol described above.

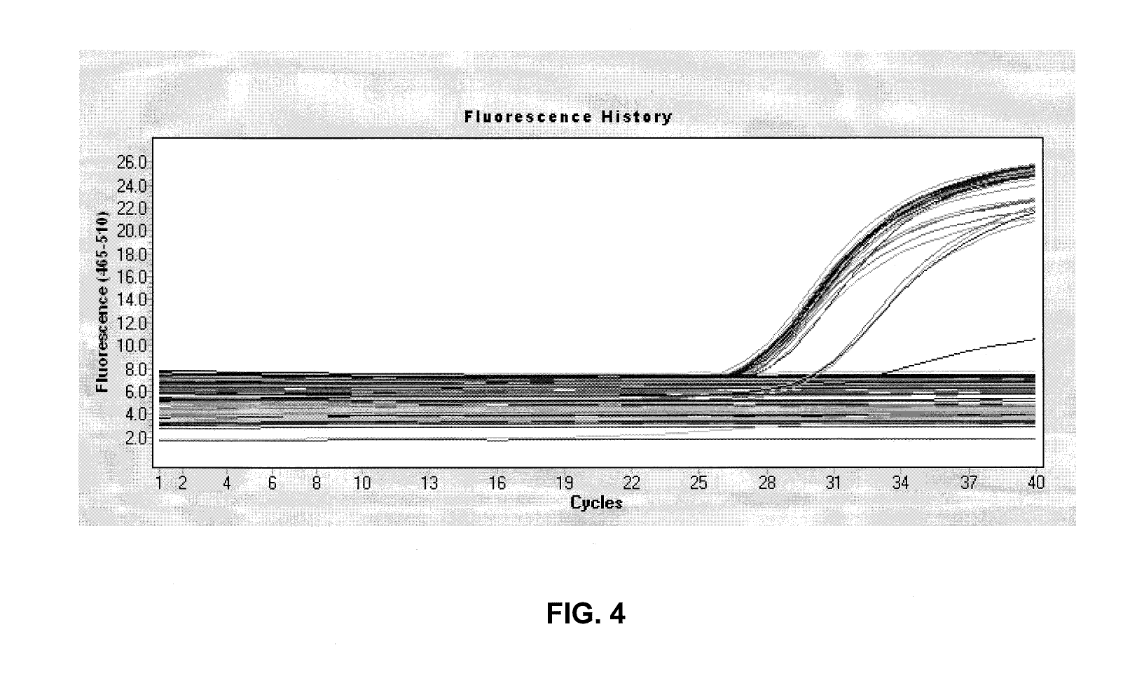

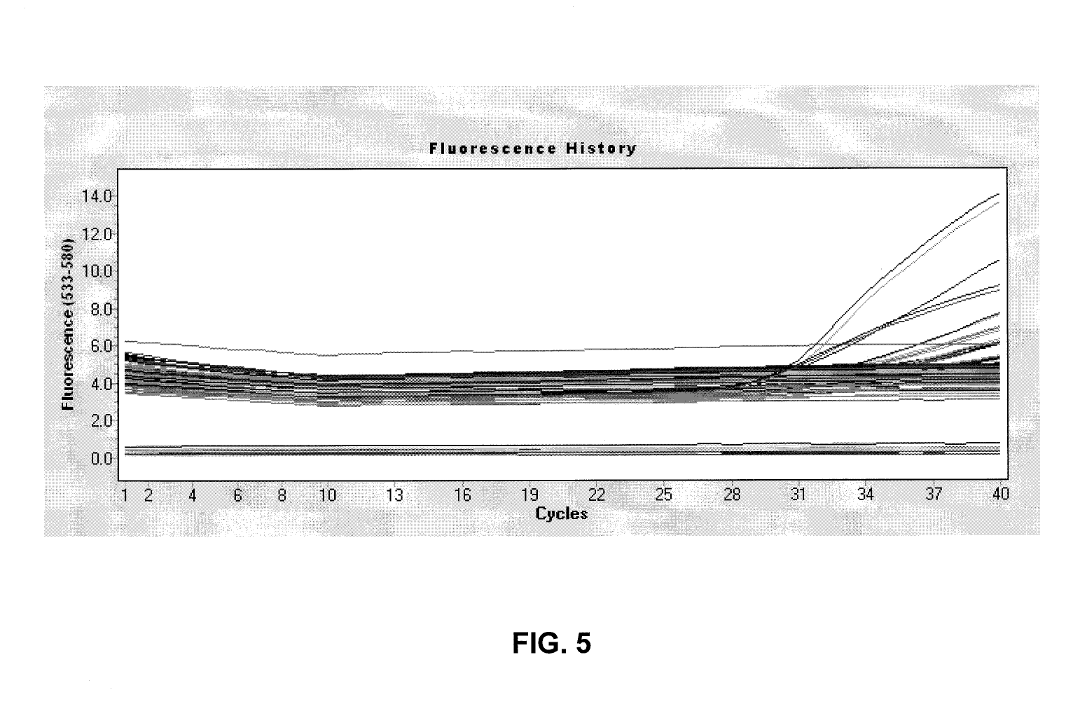

The analysis using a Roche 480 real-time thermal cycler (FIGS. 4 and 5) as well as end-point TAQMAN®-based assays indicated that all the inbred lines tested have the conserved border sequence flanking the transgene insertion. They were successfully amplified with the WT-specific primer/probe confirming that the event DAS 59122-7 assay primers and probes can be used across introgression programs for parent seed zygosity testing on finished lines.

Some of the benefits of the multiple reaction method include all the advantages of simplicity and reliability of a DNA test over ELISA test. Most importantly, it enables zygosity testing using bulk seed pools rather than ELISA testing which can only detect individual plants. In addition, this method can cut the operation cost for tester-row and ELISA testing by ten-fold. This method can also increase the sensitivity of the assay and will detect other contaminants. The multiple reaction method can also be used as an “indicator” for downstream adventitous presence (AP) testing that may be utilized for non-intended event testing and will also show the pure homozygous status of the bulk sample.

The multiple reaction method testing was proven highly sensitive in detecting presence of any hemizygous or null seed contamination in a seed lot of pure homozygous finished lines. It was shown that the multiple reaction method can detect contamination at a 1% contamination level (1 in 100 seeds). This new methodology resulted in establishing a new High Throughput Molecular Analysis (HTMA) function, better purity testing on finished lines, and can also provide a ten-fold cost savings to field operations.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A method of determining the presence or absence of an inserted nucleotide sequence at a particular insertion site in a nucleic acid, the method comprising:
isolating nucleic acid from the sample;
contacting the nucleic acid with:
a forward primer able to bind to the nucleic acid upstream of the insertion site; and
a reverse primer able to bind to the nucleic acid downstream of the insertion site;
using the primers to reproduce nucleic acids between the primers; and
analyzing the reproduced nucleic acids to determine if inserted nucleotide sequence is present or absent in the sample.

2. The method according to claim 1, further comprising contacting the nucleic acid during or after reproduction with a probe specific for a fragment amplified in the presence of the inserted nucleotide sequence.

3. The method according to claim 1, further comprising contacting the nucleic acid during or after reproduction with a probe specific for a fragment amplified in the absence of the inserted nucleotide sequence.

4. The method according to claim 2, further comprising contacting the nucleic acid during or after reproduction with a probe specific for a fragment amplified in the absence of the inserted nucleotide sequence.

5. The method according to claim 1, wherein the sample comprises more than one iteration of the particular insertion site.

6. The method according to claim 1, wherein the sample comprises nucleic acid from multiple organisms.

7. The method according to claim 5, wherein the inserted nucleotide sequence is present in less than 1% of the particular insertion sites in the nucleic acid.

8. The method according to claim 5, wherein the inserted nucleotide sequence is present in more than 99% of the particular insertion sites in the nucleic acid.

9. A method of determining the presence or absence of an inserted nucleotide sequence at a particular insertion site, the method comprising:
isolating nucleic acid from the sample;
contacting a nucleic acid with a forward primer able to bind to the nucleic acid upstream of the insertion site, and a reverse prime able to bind to the nucleic acid downstream of the insertion site;
using the primers to reproduce nucleic acids between the primers in the first portion and second portion of the nucleic acid; and
analyzing the results of the reproduction to determine if inserted nucleotide sequence is present or absent in the sample.

10. The method according to claim 9, further comprising contacting the nucleic acid during or after reproduction with a probe specific for a fragment amplified in the presence of the inserted nucleotide sequence.

11. The method according to claim 9, further comprising contacting the nucleic acid during or after reproduction with a probe specific for a fragment amplified in the absence of the inserted nucleotide sequence.

12. The method according to claim 10, further comprising contacting the nucleic acid during or after reproduction with a probe specific for a fragment amplified in the absence of the inserted nucleotide sequence.

13. The method according to claim 9, wherein the sample comprises more than one iteration of the particular insertion site.

14. The method according to claim 9, wherein the sample comprises nucleic acid from multiple different organisms.

15. The method according to claim 13, wherein the inserted nucleotide sequence is present in less than 1% of the particular insertion sites in the nucleic acid.

16. The method according to claim 13, wherein the inserted nucleotide sequence is present in more than 99% of the particular insertion sites in the nucleic acid.

17. The method according to claim 13, wherein the method is used to determine zygosity using a bulked tissue sample.

18. The method according to claim 13, wherein the method is used to determine contamination of any unintended transgenes.

19. The method according to claim 13, wherein the method is used an in indicator of adventitious event presence/absence in a bulked sample.

20. The method according to claim 13, wherein the method is used to determine contamination of any non-transgenic lines.
